(19) United States
(12) Patent Application Publication
(10) Pub. No.: US 2020/0023174 A1
(43) Pub. Date: Jan. 23, 2020

(54) MICRO-NEEDLE DEVICE, METHOD FOR MANUFACTURING THE SAME, AND METHOD FOR MANUFACTURING MICRO-NEEDLE MOLD

(71) Applicant: Tamkang University, New Taipei City (TW)

(72) Inventors: Ming-Kai Chern, New Taipei City (TW); Man Piu Chan, New Taipei City (TW); Wen-Chi Chou, New Taipei City (TW); Sey-En Lin, New Taipei City (TW); Hun-Boa Wang, New Taipei City (TW); Bo-Cheng Wang, New Taipei City (TW)

(73) Assignee: Tamkang University, New Taipei City (TW)

(21) Appl. No.: 16/151,791

(22) Filed: Oct. 4, 2018

(30) Foreign Application Priority Data
Jul. 19, 2018 (TW) 107125028

(51) Int. Cl. A61M 37/00; A61B 34/10; B29C 33/38

(52) U.S. Cl. CPC A61M 37/0015; A61B 34/10; B29C 33/3835; B29K 2083/00; A61B 2034/105; A61B 2034/104; A61M 2037/0023; A61M 2037/0053

(57) ABSTRACT

A micro-needle device is applicable for a target tissue with a first area having a first parameter and a second area having a second parameter. The micro-needle device includes a substrate and a micro-needle array connected to the substrate. The micro-needle array includes a first micro-needle corresponding to the first area and a second micro-needle corresponding to the second area. One of the surfaces of the substrate having the micro-needle array corresponds to the surface curvature of the target tissue. The first micro-needle has a first configuration corresponding to the first parameter, and the second micro-needle has a second configuration corresponding to the second parameter and different from the first configuration. The configuration includes at least one selected from a group consisting of length, distribution density, diameter, and medicine-carrying amount of micro-needle. A method for manufacturing a micro-needle device and a method for manufacturing a micro-needle mold are also provided.

### CROSS-REFERENCE TO RELATED APPLICATION

This non-provisional application claims priority under 35 U.S.C. § 119(a) to Patent Application No. 107125028 filed in Taiwan, R.O.C. on Jul. 19, 2018, the entire contents of which are hereby incorporated by reference.

### BACKGROUND

### Technical Field

The instant disclosure relates to a micro-needle, in particular, to a micro-needle device, a method for manufacturing the same, and a method for manufacturing micro-needle mold.

### Related Art

Oral administration is a common way for supplying medicine to a human. However, due to the liver primary metabolism or dyspepsia, the time for medicine absorption may be extended and the medical effect may be worsened. Intravenous injection or other subcutaneous injection methods may be used for delivering substances into the blood. However, professional or trained personnel are required for the operation. Otherwise, several adverse reactions may occur.

Micro-needle is a new-generation transdermal drug delivery system (TDDS). The micro-needle can deliver active substances to subcutaneous tissues or bloods with certain rates in an effective manner to reduce absorption variability of substances and to maintain the concentration of the active substances in the bloods. Furthermore, micro-needle treatments are painless therapeutical procedures, so that the users are more willing to have the treatments.

### SUMMARY

However, micro-needles known to the inventor are provided on a flat plane and have less flexibility in applications. Moreover, sizes and shapes of the micro-needles known to the inventor are determined and cannot be adjusted for different users. As a result, the micro-needles known to the inventor may not provide the optimized therapeutical effect for the users.

In view of this, one embodiment of the instant disclosure provides a method for manufacturing micro-needle mold. The method comprises obtaining a skin model according to longitudinal cross-section data and transversal cross-section data, wherein the skin model comprises a first area and a second area, the first area has a first parameter, and the second area has a second parameter; obtaining a micro-needle model according to the skin model, wherein the micro-needle model comprises a micro-needle array, the micro-needle array comprises a first micro-needle and a second micro-needle, the first micro-needle corresponds to the first area and the second micro-needle corresponds to the second area, the first micro-needle has a first configuration corresponding to the first parameter, the second micro-needle has a second configuration corresponding to the second parameter and different from the first configuration, the first configuration and the second configuration comprise at least one selected from a group consisting of length, distribution density, diameter, and medicine-carrying amount of the micro-needles; and obtaining a micro-needle mold, wherein the micro-needle mold comprises a plurality of needle-receiving cavities corresponding to the micro-needle array.

Another embodiment of the instant disclosure provides a method for manufacturing micro-needle device. The method comprises obtaining a skin model according to longitudinal cross-section data and transversal cross-section data, wherein the skin model comprises a first area and a second area, the first area has a first parameter, and the second area has a second parameter; and obtaining a micro-needle device according to the skin model, wherein the micro-needle device comprises a substrate and a micro-needle array connected to the substrate, a surface of the substrate having the micro-needle array corresponds to a curvature of a model surface of the skin model, the micro-needle array comprises a first micro-needle and a second micro-needle, the first micro-needle corresponds to the first area, the second micro-needle corresponds to the second area, the first micro-needle has a first configuration corresponding to the first parameter, the second micro-needle has a second configuration corresponding to the second parameter and different from the first configuration, the first configuration and the second configuration comprise at least one selected from a group consisting of length, distribution density, diameter, and medicine-carrying amount of the micro-needles.

A further another embodiment of the instant disclosure provides a micro-needle device. The micro-needle device is applicable for a target tissue comprising a first area and a second area. The first area has a first parameter and the second area has a second parameter. The micro-needle device comprises a substrate, a micro-needle array connected to the substrate. The micro-needle array comprises a first micro-needle corresponding to the first area and a second micro-needle corresponding to the second area. A surface of the substrate having the micro-needle array corresponds to a curvature of a tissue surface of the target tissue. The first micro-needle has a first configuration corresponding to the first parameter. The second micro-needle has a second configuration corresponding to the second parameter and different from the first configuration. The first configuration and the second configuration comprise at least one selected from a group consisting of length, distribution density, diameter, and medicine-carrying amount of the micro-needles.

In one or some embodiments, the micro-needle model further comprises a substrate connected to the micro-needle array of the micro-needle model. A surface of the substrate having the micro-needle array corresponds to a curvature of a tissue surface of the target tissue. Furthermore, in one or some embodiments, the methods comprise scanning a tissue surface of the target issue to obtain the curvature by applying an optical coherence tomography scanning, and the model surface corresponds to the tissue surface. Alternatively, in one or some embodiments, the methods comprise scanning a tissue surface of the target issue to obtain the curvature by applying a three-dimensional scanning.

In one or some embodiments, the manufacturing methods further comprise obtaining the longitudinal cross-section data and the transversal cross-section data of a target tissue by using an interference scanning to the target tissue. Furthermore, in one or some embodiments, the interference scanning is an optical coherence tomography scanning.

In one or some embodiments, the skin model further comprises a plurality of first retaining units and a second retaining unit. The first area corresponds to at least one of the first retaining units. The second area corresponds to at least one of the first retaining units and corresponds to the second retaining unit. A distance between a model surface of the skin model and a first retaining unit closest to the model surface is greater than a length of the first micro-needle. A distance between the model surface and the second retaining unit is greater than a length of the second micro-needle. The length of the first micro-needle is greater than the length of the second micro-needle.

In one or some embodiments, the skin model further comprises a non-inserting area and a third retaining unit. The micro-needle array further comprises a no-micro-needle area. The non-inserting area corresponds to the no-micro-needle area. In the non-inserting area, a distance between the model surface and the third retaining unit is less than 10 mm.

In one or some embodiments, the first retaining units are subcutaneous connective tissues. The second retaining unit and the third retaining unit comprise at least one selected from a group consisting of blood vessels, glandular tissues, and lymphoid tissues.

According to one or some embodiments of the instant disclosure, a micro-needle mold can be manufactured for users with different body shapes, different portions, and different user requirements, according to the distribution of components of the target tissue of each of the users. Then, a micro-needle device with different lengths and thicknesses micro-needles can be provided. The micro-needles of the micro-needle device corresponds to the target tissue to have different configurations, so that, for meeting users requirements, the micro-needles may have different lengths, distribution densities, diameters (thickness), medicine-carrying amounts (the amounts of active substance(s) carried by the micro-needle).

### BRIEF DESCRIPTION OF THE DRAWINGS

The disclosure will become more fully understood from the detailed description given herein below for illustration only, and thus not limitative of the disclosure, wherein:

FIG. 1 illustrates a flowchart of a method for manufacturing micro-needle mold according to a first embodiment of the instant disclosure;

FIG. 2 illustrates a perspective view of a skin model according to one embodiment of the instant disclosure;

FIG. 3 illustrates a perspective view of a micro-needle model according to one embodiment of the instant disclosure;

FIG. 4 illustrates a perspective view of a micro-needle mold according to one embodiment of the instant disclosure;

FIG. 5 illustrates a flowchart of a method for manufacturing micro-needle mold according to a second embodiment of the instant disclosure;

FIG. 6 illustrates a flowchart of a method for manufacturing micro-needle mold according to a third embodiment of the instant disclosure;

FIG. 7 illustrates a flowchart of a method for manufacturing micro-needle device according to an exemplary embodiment of the instant disclosure; and

FIG. 8 illustrates a perspective view of a micro-needle device according to an exemplary embodiment of the instant disclosure.

### DETAILED DESCRIPTION

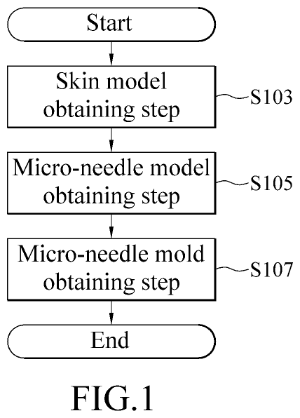

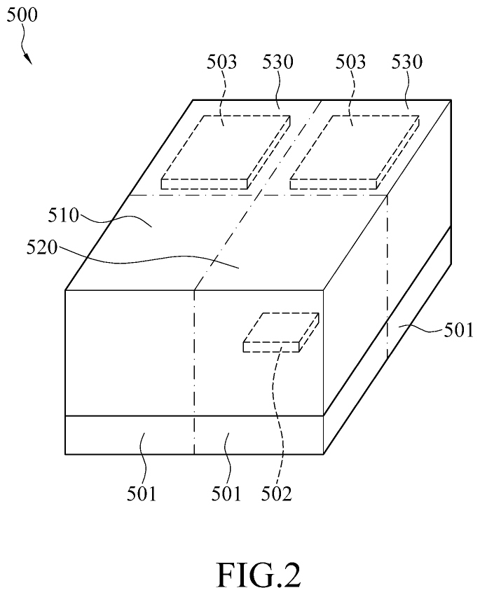

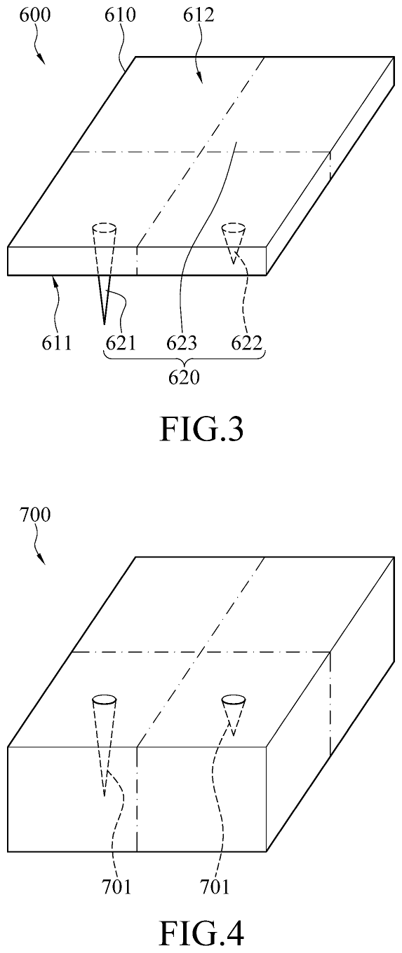

Please refer to FIG. 1, illustrating a flowchart of a method for manufacturing micro-needle mold according to a first embodiment of the instant disclosure. Please further refer to FIGS. 2 to 4. FIG. 2 illustrates a perspective view of a skin model according to one embodiment of the instant disclosure, FIG. 3 illustrates a perspective view of a micro-needle model according to one embodiment of the instant disclosure, and FIG. 4 illustrates a perspective view of a micro-needle mold according to one embodiment of the instant disclosure. As shown in FIGS. 1 to 4, a method for manufacturing a micro-needle mold comprises a skin model obtaining step S103, a micro-needle model obtaining step S105, and a micro-needle mold obtaining step S107. These steps are described as below.

Firstly, the method has the skin model obtaining step S103: obtaining a skin model according to longitudinal cross-section data and transversal cross-section data. As shown in FIG. 2, the skin model 500 comprises a first area 510 and a second area 520. The first area 510 has a first parameter, and the second area 520 has a second parameter.

The longitudinal cross-section data and the transversal cross-section data are obtained from a scanned tissue (hereinafter, target tissue). The longitudinal cross-section data and the transversal cross-section data may be presented as images. In this embodiment, the longitudinal cross-section data is at least one longitudinal cross-section image of the target tissue, and the transversal cross-section data is a plurality of transversal cross-section images of the target tissue. Furthermore, the longitudinal cross-section image is obtained by sectioning the target tissue along a direction of a lateral view of the target tissue, and the transversal cross-section images are obtained by sectioning the target tissue along a direction of a top view of the target tissue. The distribution of components of the target tissue can be obtained according to the longitudinal cross-section data and the transversal cross-section data. Taking the skin model shown in FIG. 2 as an example, first retaining units 501 are at bottom layers of each of the blocks of the skin model 500. A second retaining unit 502 is at the bottom-right block of the skin model 500. As mentioned, the upper-right block and the upper-left block of the skin model also have the first retaining units 501. Moreover, the upper-right block and the upper-left block of the skin model 500 further have a third retaining unit 503 on the model surface of the skin model 500. In one embodiment, the first retaining units 501 are subcutaneous connective tissues, and the second retaining unit 502 and the third retaining unit 503 comprise at least one selected from a group consisting of blood vessels, glandular tissues, and lymphoid tissues. In other words, in this embodiment, the skin model 500 has different distributions of components for different blocks, so that the skin model 500 can be further divided into different areas, and the areas have different parameters.

In this embodiment, a physical skin model is not necessary. That is, in one embodiment, the skin model can be generated in an electronic device according to the longitudinal cross-section data and the transversal cross-section data and then can be displayed on a display of the electronic device.

Next, the method has the micro-needle model obtaining step S105: obtaining a micro-needle model according to the skin model. Since the skin model 500 already provides the distribution of components of the target tissue, the micro-needle model 600 can be obtained according to the skin model 500.

It is understood that, in this embodiment, the micro-needle model 600 may be an electronic file stored in an electronic device. That is, a corresponding electronic file of the micro-needle model can be generated in the electronic device according to the electronic file of the skin model and then can be displayed on a display of the electronic device.

In this embodiment, the micro-needle model 600 may also be a physical micro-needle model. It is understood that, in this embodiment, the term “micro-needle model” is a tool for a subsequent rolling-over procedure, rather than a product applied to the users. Furthermore, the material of the micro-needle model 600 is also not limited.

In this embodiment, the micro-needle model 600 comprises a micro-needle array 620, and the micro-needle array 620 comprises a first micro-needle 621 and a second micro-needle 622. The first micro-needle 621 corresponds to the first area 610. The second micro-needle 622 corresponds to the second area 520. The first micro-needle 621 has a first configuration corresponding to the first parameter. The second micro-needle 622 has a second configuration corresponding to the second parameter and different from the first parameter. The configurations comprise at least one selected from a group of consisting of length, distribution density, diameter, and medicine-carrying amount (the amount of active substance(s) carried by the micro-needle) of micro-needles. In FIG. 3, the configurations are presented as the length of micro-needle.

It is understood that, in this embodiment, “the configuration corresponds to the parameter” indicates the configuration is matched with the parameter. For instance, the parameter is an insertable depth, and the configuration is a length of micro-needle.

In this embodiment, the number of micro-needle in each area is one, but embodiments are not limited thereto. In some embodiments, each area may be configured with several micro-needles. In these cases, the parameter may be the density of the blood vessels in an area, and the configuration may be the distribution density of micro-needles.

Similarly, in this embodiment, a physical micro-needle model is not necessary. That is, the micro-needle model can be generated in an electronic device according to the skin model and then can be displayed on a display of the electronic device.

In one or some embodiments, the skin model 500 further comprises a plurality of first retaining units 501 and a second retaining unit 502. The first area 510 corresponds to at least one of the first retaining units 501, and the second area 520 corresponds to at least one of the first retaining units 501 and corresponds to the second retaining unit 502. A distance between a model surface of the skin model 500 and the first retaining unit 501 closest to the model surface is greater than a length of the first micro-needle 621, a distance between the model surface and the second retaining unit 502 is greater than a length of the second micro-needle 622, and the length of the first micro-needle 621 is greater than the length of the second micro-needle 622. In this embodiment, “the area corresponds to the retaining unit” indicates that the retaining unit is in the area.

Therefore, when the micro-needle model 600 corresponds to the skin model 500, the first micro-needle 621 corresponds to the first area 510, and the second micro-needle 622 corresponds to the second area 520. Since the skin model 500 is formed based on the distribution of components of the target tissue, when the first area 510 of the skin model 500 has the first retaining unit 501, meaning that a corresponding portion of the target tissue cannot be inserted (e.g., the first retaining unit 501 is a subcutaneous connective tissue). Hence, a distance between the model surface of the skin model 500 and a first retaining unit 501 closest to the model surface should be greater than the length of the first micro-needle 621, so that the first micro-needle 621 is not in contact with the first retaining unit 501 when the first micro-needle 621 is inserted into the first area 510 of the skin model 500. Similarly, the second area 520 not only has the first retaining unit 501 but also the second retaining unit 502. A distance between the second retaining unit 502 and the model surface of the skin model 5000 is less than the distance between the first retaining unit 501 and the model surface of the skin model 500, and the distance between the second retaining unit 502 and the model surface of the skin model 500 is greater than the length of the second micro-needle 622, meaning that a corresponding portion of the target tissue cannot be inserted (e.g., the second retaining unit 502 is a blood vessel).

Last, the method has the micro-needle mold obtaining step S107: obtaining the micro-needle mold. The micro-needle mold 700 comprises a plurality of needle-receiving cavities 701. The needle-receiving cavities 701 correspond to the micro-needle array 620 (that is, the needle-receiving cavities 701 are used to receive the micro-needle array 620), as shown in FIG. 4.

In this embodiment, the micro-needle mold 700 may be an electronic file stored in an electronic device. That is, a corresponding electronic file of the micro-needle model can be generated in the electronic device according to the electronic file of the micro-needle model and then can be displayed on a display of the electronic device.

In this embodiment, the micro-needle mold 700 may be a physical micro-needle mold product. For example, the micro-needle mold product may be manufactured by using three-dimensional printing techniques according to the electronic file of the micro-needle mold, but embodiments are not limited thereto. The micro-needle mold product may also be manufactured by using other techniques. Moreover, in this embodiment, the material for making the micro-needle mold product may be polydimethylsiloxane (PDMS), but embodiments are not limited thereto. Furthermore, the micro-needle mold product may have structure facilitating for the subsequent demolding procedure.

Accordingly, the micro-needle mold 700 can be manufactured according to different distributions of elements of the subcutaneous target tissues for different users to meet different user requirements. Furthermore, after the micro-needle mold 700 is manufactured, carbohydrates or other proper biodegradable materials are injected into the needle-receiving cavities 701 of the micro-needle mold 700 and parameters like pressures and temperatures are adjusted for forming a micro-needle device.

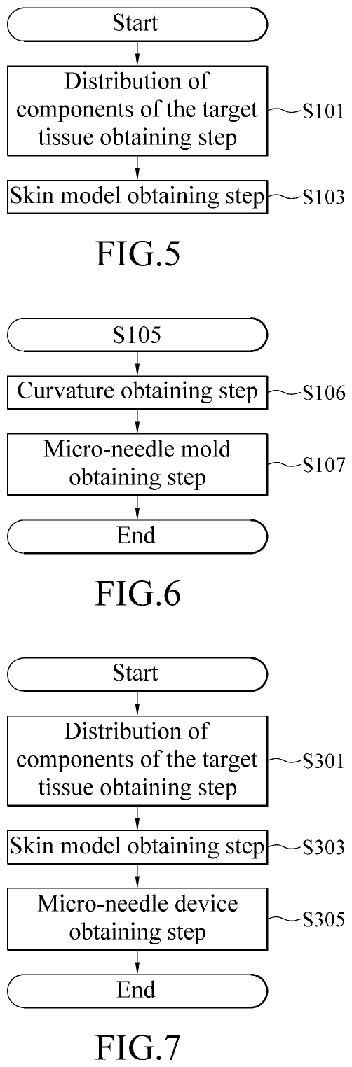

Please refer to FIG. 5, illustrating a flowchart of a method for manufacturing micro-needle mold according to a second embodiment of the instant disclosure. In one or some embodiments, the method for manufacturing micro-needle mold further comprises a distribution of components of the target tissue obtaining step S101: obtaining the longitudinal cross-section data and the transversal cross-section data of the target tissue by applying an interference scanning to the target tissue. In one embodiment, the method comprises a step of obtaining the longitudinal cross-section data and the transversal cross-section data of the target tissue by applying an optical coherence tomography scanning (hereinafter, OCT scanning) to the target tissue, but embodiments are not limited thereto.

The OCT scanning is an approach for obtaining and processing optical signals. The OCT scanning utilizes the principle of interference to scan an optical scattering medium (e.g., the target tissue) to obtain a cross-section image of the target tissue by light reflection from the target tissue in a nondestructive manner. In this embodiment, the target may be a human body, but embodiments are not limited thereto. That is, in this embodiment, the OCT technique is used to scan the human body tissue to obtain the longitudinal cross-section data and the transversal cross-section data. Furthermore, the human body tissue to be scanned may be a portion of a human's arm, but embodiments are not limited thereto.

Please refer to FIG. 2. In one or some embodiments, the skin model 500 further comprises a non-inserting area 530 and a third retaining unit 503, the micro-needle array 620 further comprises a no-micro-needle area 623, and the non-inserting area 530 corresponds to the no-micro-needle area 623. In the non-inserting area 530, a distance between the model surface of the skin model 500 and the third retaining unit 503 is less than 10 mm. Taking the skin model 500 shown in FIG. 2 as an example, the non-inserting area 530 is at the upper left block and the upper right block of the skin model 500. Further, in these blocks, the distance between the third retaining unit 503 and the model surface is less than 10 mm. That is, the third retaining unit 503 is too close to the model surface to configure the micro-needles. Hence, no-micro-needle area 623 is provided on the micro-needle array 620 to correspond to the third retaining unit 503 for mating the distribution of components of the target tissue.

Please refer to FIGS. 3 and 6. FIG. 6 illustrates a flowchart of a method for manufacturing micro-needle mold according to a third embodiment of the instant disclosure. Please refer to FIG. 3 first. In one or some embodiments, the micro-needle model 600 further comprises a substrate 610. The substrate 610 has a first surface 611 and a second surface 612 opposite to the first surface 611, and the micro-needle array 620 is connected to the first surface 611. The first surface 611 corresponds to a curvature of the model surface of the skin model 500. In other words, the surface of the substrate 610 having the micro-needle array 620 corresponds to the curvature of the model surface of the skin model 500, so that the surface can be attached on the model surface. Please further refer to FIG. 6. In this embodiment, the method for manufacturing micro-needle mold further comprises a curvature obtaining step S106: scanning a tissue surface of the target issue to obtain the curvature. The model surface corresponds to the tissue surface. In one embodiment, the curvature is obtained by applying the optical coherence tomography scanning to the target tissue. In another embodiment, the curvature is obtained by applying a three-dimensional scanning to the target tissue. As compared with the curvature obtained from the optical coherence scanning, the curvature obtained from the three-dimensional scanning has a higher resolution, so that the manufactured micro-needle device can be attached on the tissue surface of the target tissue closely and properly. Thus, the micro-needle device manufactured from the method for manufacturing micro-needle mold according to one or some embodiments of the instant disclosure can be attached on the target tissue. After the micro-needle device is attached on the user, the active substance in the micro-needle mold can be delivered to proper portions of the user in an appropriate manner.

In this embodiment, since the first surface 611 of the micro-needle model 600 has the curvature, the substrate 610 of the micro-needle model 600 can be a non-planar surface. moreover, the micro-needles of the micro-needle array 620 may be arranged in a non-parallel manner, so that an angle between the first micro-needle 621 and the substrate 610 may be same as or different from an angle between the second micro-needle 622 and the substrate 610, according to the curvature of the target surface of the target tissue.

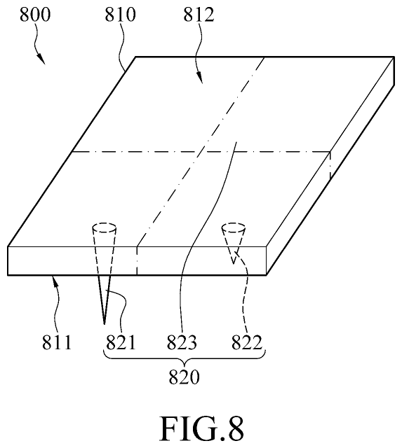

Please refer to FIGS. 7 and 8. FIG. 7 illustrates a flowchart of a method for manufacturing micro-needle device according to an exemplary embodiment of the instant disclosure. FIG. 8 illustrates a perspective view of a micro-needle device according to an exemplary embodiment of the instant disclosure. Please refer to FIGS. 2, 7, and 8. In one embodiment, a method for manufacturing micro-needle device is provided and the method comprises a distribution of components of the target tissue obtaining step S301, a skin model obtaining step S303, and a micro-needle device obtaining step S305.

Firstly, the method has the distribution of components of the target tissue obtaining step S301: obtaining longitudinal cross-section data and transversal cross-section data of a target tissue by applying an interference scanning to the target tissue. In one embodiment, the method comprises a step of obtaining the longitudinal cross-section data and the transversal cross-section data of the target tissue by applying an optical coherence tomography scanning (OCT scanning) to the target tissue, but embodiments are not limited thereto.

Next, the method has the skin model obtaining step S303: obtaining a skin model according to longitudinal cross-section data and transversal cross-section data. As shown in FIG. 2, the skin model 500 comprises a first area 510 and a second area 520. The first area 510 has a first parameter, and the second area 520 has a second parameter.

Last, the method has the micro-needle device obtaining step S305: obtaining a micro-needle device according to the skin model. In this embodiment, the micro-needle device 800 comprises a substrate 810 and a micro-needle array 820. The substrate 810 has a first surface 811 and a second surface 812 opposite to the first surface 811, and the micro-needle array 820 is connected to the first surface 811. The first surface 811 corresponds to a curvature of a model surface of the skin model 500. The micro-needle array 820 comprises a first micro-needle 821 and a second micro-needle 822. The first micro-needle 821 corresponds to the first area 510 of the skin model 500. The second micro-needle 822 corresponds to the second area 520 of the skin model 500. The first micro-needle 821 has a first configuration corresponding to the first parameter. The second micro-needle 822 has a second configuration corresponding to the second parameter and different from the first parameter. The configurations comprise at least one selected from a group of consisting of length, distribution density, diameter, and medicine-carrying amount (the amount of active substance(s) carried by the micro-needle) of micro-needles.

In this embodiment, instead of the micro-needle model for the subsequent rolling-over procedure, the micro-needle device that can be directly attached onto the user's body is manufactured. Furthermore, in this embodiment, the material for making the micro-needle device may be carbohydrates or other proper biodegradable materials. Hence, the micro-needle device can be applied on the user's target tissue and can be absorbed and degraded accordingly. Moreover, in this embodiment, the micro-needle device may be manufactured by using three-dimensional printing techniques according to the distribution of components of the target tissue, but embodiments are not limited thereto. The micro-needle device may also be manufactured by using other mold techniques.

In one or some embodiments, the curvature of the model surface is obtained by using the OCT scanning to scan the tissue surface of the target tissue, and the model surface corresponds to the tissue surface. In another embodiment, the curvature of the model surface is obtained by using the three dimensional scanning to scan the tissue surface of the target tissue, and the model surface corresponds to the tissue surface. Here, “the model surface corresponds to the tissue surface” indicates that the model surface and the tissue surface have the same profile and curvature(s).

In this embodiment, since the first surface 811 of the micro-needle device 800 has the curvature, the substrate 810 of the micro-needle device 800 can be a non-planar surface. moreover, the micro-needles of the micro-needle array 820 may be arranged in a non-parallel manner, so that an angle between the first micro-needle 821 and the substrate 810 may be same as or different from an angle between the second micro-needle 822 and the substrate 810, according to the curvature of the target surface of the target tissue.

Similarly, in one or some embodiments,

[symbol]

In one or some embodiments, the skin model 500 further comprises a plurality of first retaining units 501 and a second retaining unit 502. The first area 510 corresponds to at least one of the first retaining units 501, and the second area 520 corresponds to at least one of the first retaining units 501 and corresponds to the second retaining unit 502. The first retaining unit 501 corresponding to the second area 520 may be the same as or different from the first retaining unit 501 corresponding to the first area 510. A distance between a model surface of the skin model 500 and the first retaining unit 501 closest to the model surface is greater than a length of the first micro-needle 821, a distance between the model surface and the second retaining unit 502 is greater than a length of the second micro-needle 822, and the length of the first micro-needle 821 is greater than the length of the second micro-needle 822.

Similarly, in one or some embodiments, the skin model 500 further comprises a non-inserting area 530 and a third retaining unit 503, the micro-needle array 820 further comprises a no-micro-needle area 823, and the non-inserting area 530 corresponds to the no-micro-needle area 823. In the non-inserting area 530, a distance between the model surface of the skin model 500 and the third retaining unit 503 is less than 10 mm.

According to one or some embodiments of the instant disclosure, a micro-needle mold can be manufactured for users with different body shapes, different portions, and different user requirements, according to the distribution of components of the target tissue of each of the users. Then, a micro-needle device with different lengths and thicknesses micro-needles can be provided. The micro-needles of the micro-needle device corresponds to the target tissue to have different configurations, so that, for meeting users requirements, the micro-needles may have different lengths, distribution densities, diameters (thickness), medicine-carrying amounts (the amounts of active substance(s) carried by the micro-needle).

What is claimed is:

1. A method for manufacturing micro-needle mold, comprising:
obtaining a skin model according to longitudinal cross-section data and transversal cross-section data, wherein the skin model comprises a first area and a second area, the first area has a first parameter, and the second area has a second parameter;
obtaining a micro-needle model according to the skin model, wherein the micro-needle model comprises a micro-needle array, the micro-needle array comprises a first micro-needle and a second micro-needle, the first micro-needle corresponds to the first area and the second micro-needle corresponds to the second area, the first micro-needle has a first configuration corresponding to the first parameter, the second micro-needle has a second configuration corresponding to the second parameter and different from the first configuration, the first configuration and the second configuration comprise at least one selected from a group consisting of length, distribution density, diameter, and medicine-carrying amount of the micro-needles; and
obtaining a micro-needle mold, wherein the micro-needle mold comprises a plurality of needle-receiving cavities corresponding to the micro-needle array.

2. The method according to claim 1, further comprising obtaining the longitudinal cross-section data and the transversal cross-section data of a target tissue by applying an interference scanning to the target tissue.

3. The method according to claim 2, wherein the interference scanning is an optical coherence tomography scanning.

4. The method according to claim 1, wherein the skin model further comprises a plurality of first retaining units and a second retaining unit, the first area corresponds to at least one of the first retaining units, the second area corresponds to at least one of the first retaining units and corresponds to the second retaining unit, a distance between a model surface of the skin model and a first retaining unit closest to the model surface is greater than a length of the first micro-needle, a distance between the model surface and the second retaining unit is greater than a length of the second micro-needle, and the length of the first micro-needle is greater than the length of the second micro-needle.

5. The method according to claim 4, wherein the skin model further comprises a non-inserting area and a third retaining unit, the micro-needle array further comprises a no-micro-needle area, the non-inserting area corresponds to the no-micro-needle area; wherein, in the non-inserting area, a distance between the model surface and the third retaining unit is less than 10 mm.

6. The method according to claim 5, wherein the first retaining units are subcutaneous connective tissues, and wherein the second retaining unit and the third retaining unit comprise at least one selected from a group consisting of blood vessels, glandular tissues, and lymphoid tissues.

7. The method according to claim 1, wherein the micro-needle model further comprises a substrate connected to the micro-needle array, a surface of the substrate having the micro-needle array corresponds to a curvature of a model surface of the skin model.

8. The method according to claim 7, further comprising scanning a tissue surface of a target tissue to obtain the curvature by applying an optical coherence tomography scanning to the target tissue, wherein the model surface corresponds to the tissue surface.

9. The method according to claim 7, further comprising scanning a tissue surface of a target tissue to obtain the curvature by applying a three-dimensional scanning to the target tissue, wherein the model surface corresponds to the tissue surface.

10. A method for manufacturing micro-needle device, comprising:
obtaining a skin model according to longitudinal cross-section data and transversal cross-section data, wherein the skin model comprises a first area and a second area, the first area has a first parameter, and the second area has a second parameter; and
obtaining a micro-needle device according to the skin model, wherein the micro-needle device comprises a substrate and a micro-needle array connected to the substrate, a surface of the substrate having the micro-needle array corresponds to a curvature of a model surface of the skin model, the micro-needle array comprises a first micro-needle and a second micro-needle, the first micro-needle corresponds to the first area, the second micro-needle corresponds to the second area, the first micro-needle has a first configuration corresponding to the first parameter, the second micro-needle has a second configuration corresponding to the second parameter and different from the first configuration, the first configuration and the second configuration comprise at least one selected from a group consisting of length, distribution density, diameter, and medicine-carrying amount of the micro-needles.

11. The method according to claim 10, further comprising obtaining the longitudinal cross-section data and the transversal cross-section data of a target tissue by applying an interference scanning to the target tissue.

12. The method according to claim 11, wherein the interference scanning is an optical coherence tomography scanning.

13. The method according to claim 10, wherein the skin model further comprises a plurality of first retaining units and a second retaining unit, the first area corresponds to at least one of the first retaining units, the second area corresponds to at least one of the first retaining units and corresponds to the second retaining unit, a distance between the model surface and a first retaining unit closest to the model surface is greater than a length of the first micro-needle, a distance between the model surface and the second retaining unit is greater than a length of the second micro-needle, and the length of the first micro-needle is greater than the length of the second micro-needle.

14. The method according to claim 13, wherein the skin model further comprises a non-inserting area and a third retaining unit, the micro-needle array further comprises a no-micro-needle area, the non-inserting area corresponds to the no-micro-needle area; wherein, in the non-inserting area, a distance between the model surface and the third retaining unit is less than 10 mm.

15. The method according to claim 14, wherein the first retaining units are subcutaneous connective tissues, and wherein the second retaining unit and the third retaining unit comprise at least one selected from a group consisting of blood vessels, glandular tissues, and lymphoid tissues.

16. The method according to claim 10, further comprising scanning a tissue surface of a target tissue to obtain the curvature by applying an optical coherence tomography scanning to the target tissue, wherein the model surface corresponds to the tissue surface.

17. The method according to claim 10, further comprising scanning a tissue surface of a target tissue to obtain the curvature by applying a three-dimensional scanning to the target tissue, wherein the model surface corresponds to the tissue surface.

18. A micro-needle device applicable for a target tissue comprising a first area and a second area, the first area having a first parameter and the second area having a second parameter, the micro-needle device comprising:
a substrate; and
a micro-needle array connected to the substrate and comprising:
a first micro-needle corresponding to the first area; and
a second micro-needle corresponding to the second area;
wherein, a surface of the substrate having the micro-needle array corresponds to a curvature of a tissue surface of the target tissue, the first micro-needle has a first configuration corresponding to the first parameter, the second micro-needle has a second configuration corresponding to the second parameter and different from the first configuration, the first configuration and the second configuration comprise at least one selected from a group consisting of length, distribution density, diameter, and medicine-carrying amount of the micro-needles.

19. The method according to claim 18, wherein the skin model further comprises a plurality of first retaining units and a second retaining unit, the first area corresponds to at least one of the first retaining units, the second area corresponds to at least one of the first retaining units and corresponds to the second retaining unit, a distance between a model surface of the skin model and a first retaining unit closest to the model surface is greater than a length of the first micro-needle, a distance between the model surface and the second retaining unit is greater than a length of the second micro-needle, and the length of the first micro-needle is greater than the length of the second micro-needle.

20. The method according to claim 19, wherein the skin model further comprises a non-inserting area and a third retaining unit, the micro-needle array further comprises a no-micro-needle area, the non-inserting area corresponds to the no-micro-needle area; wherein, in the non-inserting area, a distance between the model surface and the third retaining unit is less than 10 mm.
